(19) United States
(12) Plant Patent Application Publication
(10) Pub. No.: US 2007/0271646 P1
(43) Pub. Date: Nov. 22, 2007

(54) Anagallis monelli plant named 'Wilcat Mandarin'

(76) Inventor: Rosanna Freyre, Durham, NH (US)

Correspondence Address: DEVINE, MILLIMET & BRANCH, P.A., 111 AMHERST STREET, BOX 719, MANCHESTER, NH, 03105, US

(21) Appl. No.: 11/437,314

(22) Filed: May 19, 2006

(51) Int. Cl. A01H 5/00

(52) U.S. Cl. PLT/263

(57) ABSTRACT

A new and distinct cultiva of Anagallis plant named ‘Wildcat Mandarin,’ characterized by its vigorous and outwardly spreading plant habit; freely branching growth habit; numerous single flowers that are bright orange in color with a pink center; and good garden performance.

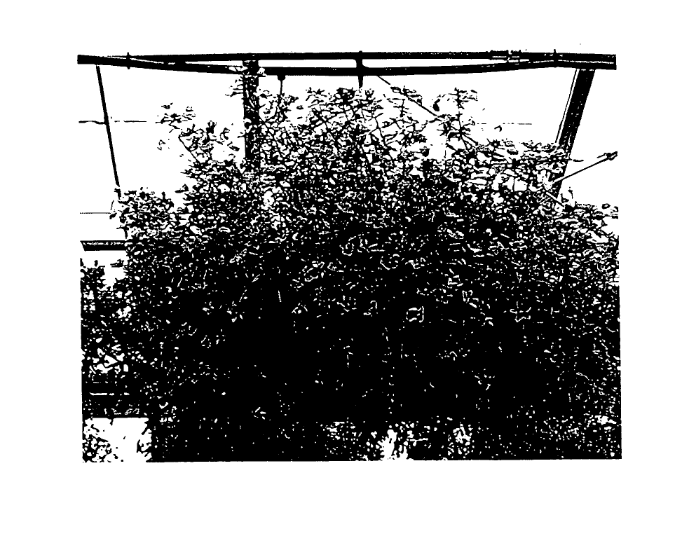

### BACKGROUND OF THE INVENTION

The present Invention relates to a new and distinct cultivar of Anagallis plant, botanically known as Anagallis monelli, previously identified with code number UNH 062-2 and hereinafter referred to by the cultivar name ‘Wildcat Mandarin’.

The new Anagallis is a product of a planned breeding program conducted by the Inventor in Durham, N.H. The objective of the breeding program is to create new compact Anagallis plants with freely and early flowering habit.

The new Anagallis originated from a hybridization made by the Inventor on Feb. 28, 2000 between two proprietary selections of Anagallis monelli, not patented, identified as code number UNH 950-2, and UNH 924-3. The new Anagallis was selected as a single plant from the resulting progeny in Durham, N.H., on the basis of its plant habit and attractive flower coloration.

Asexual reproduction of the new cultivar by terminal vegetative cuttings since Oct. 15, 2000, taken in Durham, N.H. has shown that the unique features of this new Anagallis are stable and reproduced true to type in successive generations.

### SUMMARY OF THE INVENTION

Plants of the cultivar ‘Wildcat Mandarin’ have not been observed under all possible environmental conditions. The phenotype may vary somewhat with variations in environment such as temperature, light intensity and daylength without, however, any variance in genotype.

The following traits have been repeatedly observed and are determined to be the unique characteristics of ‘Wildcat Mandarin’. These characteristics in combination distinguish ‘Wildcat Mandarin’ as a new and distinct cultivar:

1. Vigorous and outwardly spreading plant habit.

2. Freely branching growth habit:

3. Numerous single flowers that are bright orange in color, with a pink center.

4. Good garden performance.

In side-by-side comparisons conducted in Durham, N.H., plants of the new Anagallis differed from plants of Anagallis ‘Wildcat Orange’ U.S. Plant Pat. No. 15,136 P2, in the following characteristics:

1. Plants of the new Anagallis were more upright and vigorous than plants of the cultivar ‘Wildcat Orange’.

2. Plants of the new Anagallis had larger flowers than plants of the cultivar ‘Wildcat Orange’

3. Plants of the new Anagallis had orange flowers that were brighter than plants of the cultivar ‘Wildcat Orange’ and had a pink center.

4. Plants of the new Anagallis had larger leaves than plants of the cultivar ‘Wildcat Orange’.

### BRIEF DESCRIPTION OF THE PHOTOGRAPHS

The accompanying colored photographs illustrate the overall appearance of the new cultivar, showing the colors as true as it is reasonably possible to obtain in colored reproductions of this type. Colors in the photographs may differ slightly from the color values cited in the detailed botanical description which accurately describe the colors of the new Anagallis.

One photograph comprises a side perspective view of one typical 20-cm container of ‘Wildcat Mandarin’ with three plants.

The other photograph is a close-up view of typical flowers and leaves of ‘Wildcat Mandarin’.

### DETAILED BOTANICAL DESCRIPTION

The aforementioned photographs and following observations and measurements describe plants grown in Durham, N.H., in a heated polyethylene-covered greenhouse with 21° C. day/18° C. night set points. After planting rooted cuttings, plants were grown for about four months in 20-cm containers with three plants per container. Color references are made to The Royal Horticultural Society Colour Chart, 1995 Edition, except where general terms of ordinary dictionary significance are used. Color was evaluated under indirect, natural light.
Botanical classification: Anagallis monelli cultivar ‘Wildcat Mandarin’.
Parentage:
   Female parent.—Proprietary selection of Anagallis monelli identified as code number UNH 950-2, not patented.
   Male parent.—Proprietary selection of Anagallis monelli identified as code number UNH 924-3, not patented.
Propagation:
   Type cutting.—Terminal vegetative cuttings.
   Time to initiate roots.—About five days at 21° C.
   Time to develop roots.—About 15 days at 21° C.
   Root description.—Fine, fibrous; white, color 155D.
   Rooting habit.—Freely branching.
Plant description:
   Form.—Annual flowering plant; vigorous; initially upright, then semi-upright to outwardly spreading and trailing; uniformly mounded plant form. Freely branching habit with lateral branches potentially forming at very node.
   Plant height.—About 30 cm.
   Plant diameter (area of spread), single plant.—About 40 cm.
   Vigor.—Vigorous; rapid growth rate.
   Lateral branches.—Length: About 30 cm. Diameter: About 2.5 mm. Internode length: 3-4.5 cm.
   Texture.—Smooth, glabrous. Color: 144A.
   Foliage description.—Arrangement: Opposite; towards terminals, in whorls of three; simple; sessile. Length: About 2.5 cm. Width: About 8 mm. Shape: oblong to oblanceolate. Apex: Acute. Base: Truncate. Margin: Entire. Texture, upper and lower surfaces: Glabrous, smooth. Venation pattern: Pinnate. Color: Developing leaves, upper and lower surfaces: 144A. Fully expanded leaves, upper surface: 137A. Fully expanded leaves, lower surface: 137C. Venation, upper surface: 137A. Venation, lower surface: 137C.
Flower description:
   Flower type and habit.—Single star-shaped flowers; flowers face mostly upward or outward; terminal or axillary; freely flowering habit. 3-4 flowers per node; about 8 open flowers and about 12 flower buds per lateral branch at one time.
   Natural flowering season.—Plants flower from April to October in the Northern Hemisphere until frost in the autumn; flowering continuous during this period. Plants start flowering about six to eight weeks after planting rooted cuttings. Flowers not persistent.
   Flower longevity on the plant.—About four to five days.
   Fragrance.—None detected.

Flower size.—Diameter: 2.7-2.9 cm. Depth (height): About 5 mm.
Flower buds.—Length: About 7 mm. Diameter: About 4 mm. Shape: Ovate. Color: 34A.
Petals.—Quantity/arrangement: Five petals arranged in a single whorl. Length: About 1.7 cm. Width: About 1.7 cm when extended. Shape: Rhomboid, edges curl upwards. Apex: Rounded. Base: Wedged. Margin: With tiny teeth. Texture, upper and lower surfaces: Smooth, velvety. Color: When opening, upper surface: 32A. When opening, lower surface: 34A. Fully opened, upper surface: 33B; base, 67A. Fully opened, lower surface: 33C; base, 67A.
Sepals.—Arrangement/appearance: Five sepals arranged in a single whorl between the petals; star-shaped. Length: About 7 mm. Width: About 1.2 mm. Shape: Narrowly lanceolate. Apex: Acute. Base: Truncate. Margin: Entire. Texture, upper and lower surfaces: Smooth, glabrous. Color, upper and lower surfaces: 137A.
Pedicels.—Length: About 4.5 cm. Width: About 1 mm. Angle: About 45° from the main stem. Strength: Moderately strong. Texture: Smooth, glabrous. Color: 146B.
Reproductive organs.—Stamens: Quantity per flower: Five. Anther shape: Sickle-shaped. Anther length: About 2 mm. Anther width: About 1 mm. Anther color: 12A. Pollen amount: Moderate. Pollen color: 12A. Filament length: About 4 mm. Filament width: About 1 mm. Pistils: Quantity per flower: One. Pistil length: About 7 mm. Style length: About 5 mm. Style color: 67A. Stigma shape: Rounded. Stigma color: 146C. Ovary color: 146C.
Seed/fruit.—Seed and/or fruit production is moderate under open pollination with other Anagallis plants.
Disease/pest resistance: Plants of the new Anagallis have not been noted to be resistant to pathogens or pests common to Anagallis.
Garden performance: Plants of the new Anagallis have been observed to have good garden performance and tolerate temperatures from −5 to 40° C.

What is claimed is:

1. A new and distinct cultivar of Anagallis plant named ‘Wildcat Mandarin,’ as illustrated and described.
